(19) United States
(12) Patent Application Publication
(10) Pub. No.: US 2025/0367340 A1
(43) Pub. Date: Dec. 4, 2025

(54) DUAL ANTIBACTERIAL HEMOSTATIC BIO-BASED POLYURETHANE HYDROGEL CONTAINING NATURAL ANTIBACTERIAL COMPONENTS

(71) Applicant: Hefei University of Technology, Hefei (CN)

(72) Inventors: Yunsheng DING, Hefei (CN); Junjie WANG, Hefei (CN); Yufeng KANG, Hefei (CN); Zhixiu LIU, Hefei (CN); Haibing WEI, Hefei (CN); Jiaji ZHU, Hefei (CN); Jinghua DU, Hefei (CN)

(73) Assignee: Hefei University of Technology, Hefei (CN)

(21) Appl. No.: 19/208,645

(22) Filed: May 15, 2025

(30) Foreign Application Priority Data
May 30, 2024 (CN) 202410689917.0

(51) Int. Cl. A61L 15/64; A61L 15/20; A61L 15/26; A61L 15/44

(52) U.S. Cl. CPC A61L 15/64; A61L 15/20; A61L 15/26; A61L 15/44; A61L 2300/406; A61L 2300/418

(57) ABSTRACT

A dual antibacterial, hemostatic, bio-based polyurethane hydrogel containing natural antibacterial components is provided. The hydrogel is prepared using a one-step, solvent-free, catalyst-free method. The raw materials include: biodegradable polyester polyol; polyethylene glycol; bio-based diisocyanate; a hydrophilic alcohol chain extender; curcumin; glycerol; deionised water; and Panax notoginseng extract. In the hydrogel: Curcumin promotes the physical and chemical crosslinking of the polyurethane hydrogel, improving its antioxidant and antibacterial properties. The active components of Panax notoginseng saponins are loaded into the hydrogel via hydrogen bonding between the sulfonic acid group, the carboxyl group on the polyurethane molecular chain, and the saponins. This gives the hydrogel hemostatic and antibacterial properties. At the same time, the hydrogel releases the drug slowly when it absorbs weakly alkaline wound exudate. The bio-based polyurethane hydrogel prepared using this method is biodegradable and has good mechanical properties, rapid haemostatic ability, and long-term antibacterial properties.

### CROSS REFERENCE TO THE RELATED APPLICATIONS

This application is based upon and claims priority to Chinese Patent Application No. 202410689917.0, filed on May 30, 2024, the entire contents of which are incorporated herein by reference.

### TECHNICAL FIELD

The invention belongs to the field of polymer science and technology, and specifically relates to a dual antibacterial hemostatic bio-based polyurethane hydrogel containing natural antibacterial components.

### BACKGROUND

Medical wound dressings cover the wounded skin to protect wounds and prevent secondary skin damage. They also provide an environment is favorable for wound healing, and are an important biomedical material. Commonly used dressings mainly include gauze and hydrogel. A hydrogel dressing is a three-dimensional polymer gel network with better performance than traditional gauze dressing. It is primarily formed by crosslinking a hydrophilic polymer through physical or chemical action. Modern wound healing theory posits that wet healing is fundamental to the process. Therefore, keeping the wound wet during wound healing is an inevitable requirement for functional dressings. Hydrogels are very soft due to their large amount of water and can be used as excellent biomedical materials, especially new wound dressings. When used in wound dressings, hydrogels can reduce the infection of external bacteria and microorganisms on the wound, effectively prevent the loss of body fluids, and transmit oxygen to the wound. They also maintain a certain degree of moisture, thereby accelerating wound healing.

For thousands of years, the practical value of Chinese medicine in the prevention and treatment of various diseases has been proven, with Panax notoginseng being one of the most commonly used Chinese herbal medicines. It has anti-inflammatory properties and promotes angiogenesis and wound healing. Curcumin, on the other hand, is a low-molecular-weight, natural, hydrophobic polyphenol extracted from the dried rhizome of turmeric. It exhibits a variety of therapeutic properties, including anti-oxidant, anti-bacterial, anti-infective, anti-inflammatory and anti-fibrotic effects, and is widely used in biomedical research. Effective ingredients with antibacterial and haemostatic functions can be extracted from plants and loaded into medical dressings to endow them with functionality. However, the performance of medical dressings depends not only on the functional materials they contain, but also on the structure of the dressing's carrier material. Therefore, combining the advantages of functional and base materials can meet the repair requirements of complex wounds.

### SUMMARY

The invention provides a dual antibacterial hemostatic bio-based polyurethane hydrogel containing natural antibacterial components.

The purpose of the invention is realized by the following technical schemes:

A dual antibacterial hemostatic bio-based polyurethane hydrogel containing natural antibacterial components, including the following raw materials parts by weight: 40.0-60.0 parts of biodegradable polyester polyol, 40.0-60.0 parts of polyethylene glycol, 24.9 parts of bio-based diisocyanate, 1.3 parts of hydrophilic alcohol chain extender, 0.1-3.7 parts of curcumin, 2.0-2.5 parts of glycerol, 180.0-200.0 parts of deionized water, and 20.0 parts of Panax notoginseng extract.

The invention relates to a method of preparation a dual antibacterial hemostatic bio-based polyurethane hydrogel containing natural antibacterial components. This method includes the following steps:
(1) Drying the biodegradable polyester polyol and polyethylene glycol at 110-130° C. and −0.1 MPa for 1-2 h, and then cooling to room temperature and sealing for storage to obtain a dehydrated biodegradable polyester polyol and a dehydrated polyethylene glycol;
(2) Mixing the dehydrated biodegradable polyester polyol and dehydrated polyethylene glycol obtained in Step (1) with a hydrophilic alcohol chain extender, curcumin, and glycerol, and then stirring at 70-90° C. for 1-2 h, a stirring speed is 200-500 rpm to obtain a uniform mixture.
(3) Obtaining a bio-based polyurethane slurry by adding bio-based diisocyanate to the uniform mixture prepared in Step (2), stirring at 70-90° C. for 2-4 h, and stirring at 200-500rpm.
(4) Solidifying the bio-based polyurethane slurry prepared in Step (3) in a vacuum oven at 80-100° C., and then immersing in deionized water to obtain a bio-based polyurethane hydrogel.
(5) Immersing the bio-based polyurethane hydrogel obtained in Step (4) in liquid nitrogen for 10-15 min, and then vacuum freeze-drying at −20-40° C. for 24-48 h to obtain a porous bio-based polyurethane hydrogel.
(6) Immersing the porous bio-based polyurethane hydrogel obtained in Step (5) in the Panax notoginseng extract for 3 h, so that the Panax notoginseng extract is dispersed in a matrix, and then vacuum freeze-drying at −20-40° C. for 24-48 h to obtain the dual antibacterial hemostatic bio-based polyurethane hydrogel containing natural antibacterial components.

Furthermore, the biodegradable polyester polyol is at least one of the biodegradable polycarbonate diol, biodegradable polylactic acid diol, and biodegradable polycaprolactone diol, and a number-average molecular weight of the biodegradable polyester polyol is 1000-3000 g/mol. The selected biodegradable polyester polyols have good biocompatibility and biodegradability. The sample made of its molecular weight in this range are flexible and can be used as wound dressings to adapt to the deformation caused by movement.

Furthermore, the number-average molecular weight of the polyethylene glycol is 1000-3000 g/mol. The selected molecular weight can provide good hydrophilicity.

Furthermore, the bio-based diisocyanate is at least one of lysine diisocyanate and pentamethylene diisocyanate. The selected bio-based isocyanate is derived from animal and plant resources, it has good biocompatibility and can be biodegradable.

Furthermore, the hydrophilic alcohol chain extender is a compound with the following structural formula including a hydrophilic group, where R is a linear or branched C3-C4 alkyl group:

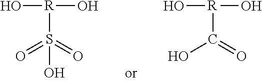

The above structural compounds have good hydrophilic groups, the hydrophilic groups can not only form hydrogen bonds with water molecules, which helps to improve water absorption, but also form hydrogen bonding interactions with drugs, thereby adsorbing and releasing drugs.

Furthermore, the curcumin has the following structure:

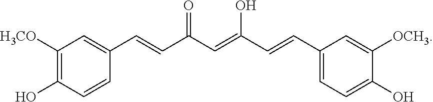 [SMILES: COc1cc(/C=C/C(=O)/C=C(O)/C=C/c2ccc(O)c(OC)c2)ccc1O]

This curcumin structure contains three functional hydroxyl groups. In the process of synthesising polyurethane, it acts as a cross-linking agent alongside glycerol. This promotes the physical and chemical cross-linking of the matrix while maintaining its antibacterial properties and endowing it with long-term antibacterial ability.

Furthermore, the Panax notoginseng extract is composed of 5.0-10.0% Panax notoginseng saponins and 90.0-95.0% deionized water.

The dual antibacterial hemostatic bio-based polyurethane hydrogel containing natural antibacterial components prepared by the invention can be used to wound dressings.

Compared with the existing technology, the beneficial effect of the invention is reflected in:
1. The invention uses biodegradable polyester polyols and hydrophilic diols as the soft segments and bio-based diisocyanates as the hard segments to prepare bio-based polyurethane hydrogels, the raw material source is bio-based and biodegradable, which is a new material that meets the requirements of sustainable development.
2. The invention adopts a one-step synthesis of bio-based polyurethane, the process is simple, and does not add any solvent or catalyst, the raw materials used are non-toxic and harmless to organisms, and can be used to prepare medical materials such as wound dressings.
3. The mechanical properties of the polyurethane hydrogel prepared by the invention can be adjusted by adjusting the ratio between the soft and hard segments, and the added natural curcumin is partially covalently bound to the macromolecular chains that make up the matrix, and partially dispersed in the matrix through physical and chemical action, while promoting the physical and chemical cross-linking of the polyurethane hydrogel, it improves its antibacterial ability and the stability of the network structure, so it has good mechanical stability in the swelling equilibrium state, and can be used to prepare wound dressings with excellent mechanical properties, which can significantly prolong the service life of the medical gel and has good practicability.
4. The polyurethane hydrogel prepared by the invention has a porous structure, which releases the Chinese herbal medicine extract while absorbing the wound blood, and can reach the adsorption swelling balance in a short time, with rapid hemostasis and debridement functions, and the wound contact surface can always maintain a moist environment, which is conducive to wound recovery. At the same time, the presence of a porous structure can promote the degradation of the bio-based polyurethane hydrogel.
5. The antibacterial properties of the polyurethane hydrogel prepared by the invention are derived from the natural antibacterial agent component and the Chinese herbal medicine extract in the hydrogel network structure. Compared with antibiotics with a single mode of action, Chinese herbal medicine is not only derived from natural plants and environmentally friendly, but also can reduce the generation of antibiotic resistance. The active components of Panax notoginseng are loaded into the hydrogel by hydrogen bonding between the sulfonic acid group and carboxyl group on the polyurethane molecular chain and the hydroxyl group in Panax notoginseng, giving it hemostatic and antibacterial effects. In addition, the hydrogel has a unique porous structure, when used as a wound dressing, while absorbing the wound exudate, the weakly alkaline exudate deprotonates the carboxyl and sulfonic acid groups in the hydrogel, eliminating the hydrogen bonding between the hydrogel and curcumin to achieve the release of the drug and achieve the adsorption swelling equilibrium in a short time. This excellent rapid water absorption ability can quickly absorb the water in the blood, which is conducive to the concentration and enrichment of coagulation factors, red blood cells, and platelets in the blood, so as to achieve the purpose of rapid hemostasis.
6. The bio-based polyurethane hydrogel prepared by the invention has biodegradability, good mechanical properties, rapid hemostasis ability, and double long-term antibacterial effect, compared with the medical gel used in the market, the bio-based polyurethane hydrogel has more obvious promotion effect on the adhesion and healing of wound coverage, which can effectively avoid the inflammation caused by bacterial infection and reduce the pain of patients.

### DETAILED DESCRIPTION OF THE EMBODIMENTS

The following is a detailed description of the specific implementation method of the invention in combination with the embodiment. The following embodiment is implemented on the premise of the technical scheme of the invention, and the detailed implementation method and specific operation process are given, but the protection scope of the invention is not limited to the following embodiment.

All raw materials in the following examples are available on the market.

The specific preparation method of Panax notoginseng extract used in the following examples is as follows: After drying and crushing, the roots of Panax notoginseng were screened by 800 mesh sieve, and anhydrous ethanol was added according to the mass ratio of material to liquid of 1:11. The extract was refluxed at 70° C. for 2 times, and then the two extracts were mixed and evaporated at 45° C. under reduced pressure to completely dry to obtain Panax notoginseng saponins. Then the dried total curcumin saponins were mixed with deionized water at a mass ratio of 1:9 to obtain Panax notoginseng extract.

### Example 1

A preparation method of a dual antibacterial hemostatic bio-based polyurethane hydrogel containing natural antibacterial components, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender, 0.9 parts of curcumin and 2.3 parts of glycerol were added at the same time, the mixture was stirred at 90° C. for 2 h, and the stirring speed was 200 rpm, a uniform mixture was obtained.
(3) The uniform mixture prepared in Step (2) was cooled to 80° C., and 24.9 parts of lysine diisocyanate was added, the reaction was stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.
(6) The porous bio-based polyurethane hydrogel obtained in Step (5) was immersed in 20.0 parts of Panax notoginseng extract for 3 h, so that the Panax notoginseng extract was dispersed in the matrix, and then vacuum freeze-dried at −40° C. for 24 h, finally, a dual antibacterial hemostatic bio-based polyurethane hydrogel containing natural antibacterial components was obtained.

### Example 2

A preparation method of a dual antibacterial hemostatic bio-based polyurethane hydrogel containing natural antibacterial components, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender, 1.8 parts of curcumin and 2.1 parts of glycerol were added at the same time, the mixture was stirred at 90° C. for 2 h, and the stirring speed was 200 rpm, a uniform mixture was obtained.
(3) The uniform mixture prepared in Step (2) was cooled to 80° C., and 24.9 parts of lysine diisocyanate was added, the reaction was stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.
(6) The porous bio-based polyurethane hydrogel obtained in Step (5) was immersed in 20.0 parts of Panax notoginseng extract for 3 h, so that the Panax notoginseng extract was dispersed in the matrix, and then vacuum freeze-dried at −40° C. for 24 h, finally, a dual antibacterial hemostatic bio-based polyurethane hydrogel containing natural antibacterial components was obtained.

### Example 3

A preparation method of a dual antibacterial hemostatic bio-based polyurethane hydrogel containing natural antibacterial components, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender, 2.8 parts of curcumin and 2.0 parts of glycerol were added at the same time, the mixture was stirred at 90° C. for 2 h, and the stirring speed was 200 rpm, a uniform mixture was obtained.
(3) The uniform mixture prepared in Step (2) was cooled to 80° C., and 24.9 parts of lysine diisocyanate was added, the reaction was stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.
(6) The porous bio-based polyurethane hydrogel obtained in Step (5) was immersed in 20.0 parts of Panax notoginseng extract for 3 h, so that the Panax notoginseng extract was dispersed in the matrix, and then vacuum freeze-dried at −40° C. for 24 h, finally, a dual antibacterial hemostatic bio-based polyurethane hydrogel containing natural antibacterial components was obtained.

### Example 4

A preparation method of a dual antibacterial hemostatic bio-based polyurethane hydrogel containing natural antibacterial components, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender, 3.7 parts of curcumin and 1.8 parts of glycerol were added at the same time, the mixture was stirred at 90° C. for 2 h, and the stirring speed was 200 rpm, a uniform mixture was obtained.
(3) The uniform mixture prepared in Step (2) was cooled to 80° C., and 24.9 parts of lysine diisocyanate was added, the reaction was stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.
(6) The porous bio-based polyurethane hydrogel obtained in Step (5) was immersed in 20.0 parts of Panax notoginseng extract for 3 h, so that the Panax notoginseng extract was dispersed in the matrix, and then vacuum freeze-dried at −40° C. for 24 h, finally, a dual antibacterial hemostatic bio-based polyurethane hydrogel containing natural antibacterial components was obtained.

### Comparison Case 1

A method for preparing a porous bio-based polyurethane hydrogel includes the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylolpropionic acid chain extender and 2.5 parts of glycerol were added at the same time, the mixture was stirred at 90° C. for 2 h, and the stirring speed was controlled to 200 rpm to obtain a uniform mixture.
(3) The uniform mixture prepared in Step (2) was cooled to 80° C., and 24.9 parts of lysine diisocyanate was added, the reaction was stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.

### Comparison Case 2

A preparation method for a bio-based polyurethane hydrogel only containing curcumin, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender, 1.8 parts of curcumin and 2.1 parts of glycerol were added at the same time, the mixture was stirred at 90° C. for 2 h, and the stirring speed was 200 rpm to obtain a uniform mixture.
(3) The uniform mixture prepared in Step (2) was cooled to 80° C., and 24.9 parts of lysine diisocyanate was added. The reaction was stirred at 80° C. for 3 h, and the stirring speed was controlled at 200 rpm to obtain bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel containing only curcumin.

### Comparison Case 3

A preparation method of a bio-based polyurethane hydrogel only containing Panax notoginseng saponins, including the following steps:
(1) The biodegradable polycaprolactone diol with a number-average molecular weight of 2000 g/mol and the hydrophilic polyethylene glycol with a number-average molecular weight of 2000 g/mol were dried at 110° C. and −0.1 MPa for 2 h, and then sealed and stored for later use after cooling to room temperature, the dehydrated biodegradable polyester polyol and the hydrophilic polyethylene glycol were obtained.
(2) 50.0 parts of polycaprolactone diol and 50.0 parts of polyethylene glycol were put into the reactor, and 1.3 parts of dimethylol propionic acid chain extender and 2.5 parts of glycerol were added at the same time, the mixture was stirred at 90° C. for 2 h, and the stirring speed was 200 rpm to obtain a uniform mixture.
(3) The uniform mixture prepared in Step (2) was cooled to 80° C., 24.9 parts of lysine diisocyanate was added and stirred at 80° C. for 3 h, and the stirring speed was controlled at 200 rpm to obtain a bio-based polyurethane slurry.
(4) The bio-based polyurethane slurry obtained in Step (3) was solidified by a vacuum oven at 90° C., and then soaked in 200.0 parts of deionized water to obtain a bio-based polyurethane hydrogel.
(5) The bio-based polyurethane hydrogel obtained in Step (4) was immersed in liquid nitrogen for 10 min, and then vacuum freeze-dried at −40° C. for 24 h to obtain a porous bio-based polyurethane hydrogel.
(6) The porous bio-based polyurethane hydrogel obtained in Step (5) was immersed in 20.0 parts of Panax notoginseng extract for 3 h, so that the Panax notoginseng extract was dispersed in the matrix, and then vacuum freeze-dried at −40° C. for 24 h, finally, a bio-based polyurethane hydrogel containing only Panax notoginseng saponins was obtained.

The following performance tests were carried out on the bio-based polyurethane hydrogels prepared by comparison cases 1-3 and examples 1-4:

### 1. Swelling Performance Test

The swelling properties of the bio-based polyurethane hydrogels prepared by Examples 1-4 and Comparison case 1 were tested, the specific method was as follows: the dry bio-based polyurethane hydrogel (i.e., the hydrogel obtained in Step (5)) that was not immersed in the Panax notoginseng extract was cut into small pieces, weighed, and the mass was recorded as mo. The above bio-based polyurethane hydrogel pieces were immersed in deionized water at 25° C., the hydrogel was taken out at regular intervals, and the excess water on the surface was removed with filter paper to obtain a hydrogel with a mass of m. The swelling rates of bio-based hydrogels at different time intervals were calculated by Eq. (1). Three parallel samples were set for each result, and the results are shown in Table 1.

$$Swelling rate ( \% ) = ( m - {m}_{0} ) / {m}_{0} \times 100 \% \qquad ( 1 )$$

TABLE 1
Swelling properties of bio-based polyurethane
hydrogels prepared in each example

| | Time | | | | | | | |
|---|---|---|---|---|---|---|---|---|
| Sample | 1(h) | 2(h) | 3(h) | 4(h) | 5(h) | 7(h) | 9(h) | 12(h) |
| Example 1 | 171% | 198% | 205% | 206% | 206% | 206% | 206% | 206% |
| Example 2 | 170% | 197% | 207% | 207% | 207% | 207% | 207% | 207% |
| Example 3 | 174% | 208% | 215% | 216% | 216% | 216% | 216% | 216% |
| Example 4 | 151% | 183% | 198% | 200% | 200% | 200% | 200% | 200% |
| Comparison | 187% | 226% | 229% | 229% | 229% | 229% | 229% | 229% |
| case 1 | | | | | | | | |

### 2. Mechanical Properties Test

The tensile strength was tested according to the standard GB/T 1040.2-2006 and the compressive strength was tested according to the standard GB/T 1041-2008, as follows: The mechanical properties of the samples were tested by the CMT4304 universal testing machine, the tensile speed was 100 mm/min and the compression speed was 10 mm/min, each sample was tested three times in parallel at different positions, and the average value was taken. The results are shown in Table 2.

TABLE 2
Mechanical properties of bio-based polyurethane
hydrogels prepared in each example

| | Tensile | Tenacity | Compressive |
|---|---|---|---|
| Samples | strength(MPa) | (MJ/m³) | strength(MPa) |
| Example 1 | 4.91 ± 0.55 | 18.78 ± 0.26 | 5.45 ± 0.36 |
| Example 2 | 6.59 ± 0.67 | 28.36 ± 0.56 | 12.31 ± 0.23 |
| Example 3 | 5.87 ± 0.45 | 23.27 ± 0.62 | 3.41 ± 0.34 |
| Example 4 | 2.36 ± 0.63 | 11.13 ± 0.32 | 3.13 ± 0.65 |
| Comparison | 3.37 ± 0.32 | 8.67 ± 0.88 | 2.71 ± 0.33 |
| case 1 | | | |
| Comparison | 6.12 ± 0.24 | 25.16 ± 0.43 | 12.11 ± 0.13 |
| case 2 | | | |
| Comparison | 3.15 ± 0.34 | 8.36 ± 0.33 | 2.61 ± 0.53 |
| case 3 | | | |

### 3. Antibacterial Performance Test

According to QB/T4341-2012, the antibacterial rates of the bio-based polyurethane hydrogel prepared by Comparison cases 1-3 and examples 1-4 against Escherichia Coli and Staphylococcus Aureus were tested. The results are shown in Table 3.

TABLE 3
Antibacterial properties of the bio-based polyurethane
hydrogels prepared in each implementation example

| | Antibacterial rate | Antibacterial rate |
|---|---|---|
| Samples | of E. coli(%) | of S. aureus(%) |
| Example 1 | 99.1% | 99.1% |
| Example 2 | 99.9% | 99.9% |
| Example 3 | 97.3% | 97.3% |
| Example 4 | 96.5% | 96.5% |
| Comparison | 0 | 0 |
| case 1 | | |
| Comparison | 90.9% | 90.9% |
| case 2 | | |
| Comparison | 93.9% | 93.9% |
| case 3 | | |

### 4. Cytotoxicity Test

The cytotoxicity test was used to determine the cell viability of the bio-based polyurethane hydrogels prepared by Comparison cases 1-3 and Examples 1-4, the specific method was: the hydrogel was sterilized with ultraviolet light for 2 h, and then immersed in the extracted complete DMEM medium for two days. DMEM and L929 mouse fibroblasts were used as blank control and model cells, respectively. The DMEM containing the extract was incubated with fibroblasts at 37° C. for 3 days. After incubation, the cell viability was detected by MTT assay. The light absorption value at the wavelength of 570 nm was detected by enzyme-linked immunosorbent assay, which could indirectly reflect the number of living cells. Cell viability=(A1/A2)×100%; A1 is the absorbance value of the cells treated with the extract; A2 is the absorbance value of the cells in the blank control group. The results are shown in Table 4.

TABLE 4
Cell viability of bio-based polyurethane
hydrogels prepared in each embodiment

| Samples | Cell viability(%) |
|---|---|
| Example 1 | 98% ± 1.5% |
| Example 2 | 101% ± 0.8% |
| Example 3 | 97% ± 2.4% |
| Example 4 | 95% ± 1.2% |
| Comparison | 80% ± 2.1% |
| case 1 | |
| Comparison | 94% ± 1.3% |
| case 2 | |
| Comparison | 95% ± 2.3% |
| case 3 | |
| Blank | 100% |
| control group | |

### 5. Degradation Performance Test

The degradation performance of the bio-based polyurethane hydrogels prepared by Comparison cases 1-3 and Examples 1-4 was tested. The specific method was as follows: 0.1 g of dry bio-based polyurethane hydrogel (i.e., the hydrogel obtained in Step (5)) was immersed in PBS solution containing 1 mg/mL porcine pancreatic lipase (pH=7.4), placed in an oven at 37° C., and weighed every four weeks. The PBS solution was updated weekly, and the pH value was kept unchanged. The detection process included rinsing the sample with distilled water, drying it in a vacuum oven at 40° C. for 24 hours, and then weighing. The degradation of hydrogels at different times was observed to determine the degradation ability of bio-based polyurethane hydrogels. The results are shown in Table 5.

TABLE 5
Degradation properties of the bio-based polyurethane
hydrogels prepared in each example

| Samples | 4 weeks(%) | 8 weeks(%) | 12 weeks(%) |
|---|---|---|---|
| Example 1 | 2.8% | 3.6% | 4.8% |
| Example 2 | 2.1% | 3.1% | 3.9% |
| Example 3 | 3.6% | 7.8% | 13.9% |
| Example 4 | 5.4% | 8.3% | 15.6% |
| Comparison | 7.3% | 15.4% | 25.3% |
| case 1 | | | |
| Comparison | 2.1% | 3.1% | 3.9% |
| case 2 | | | |
| Comparison | 6.9% | 14.6% | 23.8% |
| case 3 | | | |

### 6. Hemostatic Performance Test

200-250g rats were selected and anesthetized with 1 mL 10% chloral hydrate solution, after that, a 2 cm wound was cut on the thigh of the rat to expose the femoral artery, then, the wound was cut off with a scalpel, and a series of bio-based polyurethane hydrogels were attached to the wound. The bleeding time was observed when there was no bleeding. At the end of the experiment, the rats were euthanized. The results are shown in Table 6.

TABLE 6
Hemostatic properties of bio-based polyurethane
hydrogels prepared in each example

| | Hemostatic |
|---|---|
| Samples | time(s) |
| Example 1 | 30 |
| Example 2 | 23 |
| Example 3 | 28 |
| Example 4 | 34 |
| Comparison | 50 |
| case 1 | |
| Comparison | 43 |
| case 2 | |
| Comparison | 39 |
| case 3 | |
| Blank | 160 |
| control group | |

### 7. Wound Healing Performance Test

The wound healing performance of the bio-based polyurethane hydrogel prepared by the Comparison case 1-3 and Examples 1-4 was tested. The specific method was as follows: a wound of about 1 cm was drawn on the back of the mouse, and the wound was treated with a dressing, the dressing was replaced every other day and HE staining was used to observe inflammation. The results are shown in Table 7.

TABLE 7
Healing properties of bio-based polyurethane
hydrogels prepared in each example

| | | Whether |
|---|---|---|
| | Healing | inflammation |
| Samples | period(day) | occurs |
| Example 1 | 9 | No |
| Example 2 | 7 | No |
| Example 3 | 11 | No |
| Example 4 | 13 | No |
| Comparison | 20 | Yes |
| case 1 | | |
| Comparison | 17 | No |
| case 2 | | |
| Comparison | 15 | No |
| case 3 | | |

It can be seen from Table 1 to Table 7 that the polyurethane hydrogels prepared by the methods described in Examples 1-4 are biodegradable, and the degradation rate is relatively slow, which can make the mechanical properties of polyurethane hydrogels remain unchanged for a long time. At the same time, the degradable properties of polyurethane hydrogels contribute to the long-term release of curcumin and the Chinese herbal medicine Panax notoginseng. There is no obvious biological toxicity to L929 cells, especially Example 2 promotes the proliferation of L929 cells; compared with the blank control group, the bio-based polyurethane hydrogel can significantly shorten the hemostasis time, which fully shows that the hydrogel has a rapid hemostatic effect; the polyurethane hydrogels prepared by the methods described in Examples 1-4 and Comparison case 1 have good swelling rate, and the swelling balance can be reached within 3 hours to prevent the secondary infection caused by the reflux of the exudate, the polyurethane hydrogels prepared by the methods described in Examples 1-4 can effectively promote wound healing and shorten wound healing time. Compared with Examples 1-4, curcumin is not introduced as an antibacterial agent in Comparison case 1, and the tensile strength of the samples in Comparison case 1 is greatly reduced, and the notoginseng extract is not added to the polyurethane hydrogel matrix, the obtained polyurethane hydrogel sample does not have an antibacterial effect and the wound healing rate is the slowest. For Comparison case 2, curcumin is added as an antibacterial agent, compared with Comparison case 1, the wound healing rate increases, indicating that curcumin plays a good role in promoting the efficacy of hydrogel in promoting wound healing. Compared with Examples 1-4 and Comparison case 3, the wound healing rate of the mice is slow without the introduction of Panax notoginseng extract into the hydrogel matrix in Comparison case 2, indicating that the Chinese herbal medicine Panax notoginseng extract plays a key role in promoting wound healing of the hydrogel.

In summary, the invention provides a dual antibacterial, hemostatic, bio-based polyurethane hydrogel containing natural antibacterial components. The hydrogel is prepared using a simple, one-step, solvent-free, catalyst-free process. The raw materials used are non-toxic and harmless to organisms. By adding curcumin, which has natural antibacterial properties, as an antibacterial agent, and Panax notoginseng, a Chinese herbal medicine with hemostatic properties, the hydrogel exhibits dual antibacterial and hemostatic functions, as well as being stretchable and degradable. The introduced curcumin is partially dispersed in the matrix through physical and chemical interactions, with some of it forming covalent bonds with the macromolecular chains that make up the matrix. This promotes the physical and chemical cross-linking of the polyurethane hydrogel, thereby improving its antibacterial ability. The active components of Panax notoginseng are loaded into the hydrogel via hydrogen bonding between the sulfonic acid and carboxyl groups on the polyurethane molecular chains and the hydroxyl group in Panax notoginseng, thereby providing hemostatic and antibacterial effects. The hydrogel is freeze-dried to create a porous structure that can quickly reach adsorption swelling equilibrium and achieve rapid haemostasis when used as a wound dressing. While absorbing wound exudate, the weak alkaline exudate deprotonates the hydrogel's carboxyl and sulfonic acid groups, eliminating the hydrogen bonding with curcumin to release the drug. The hydrogel wound dressing adheres well to joint wounds and has excellent antibacterial and anti-inflammatory properties. It promotes wound healing and has anti-inflammatory properties. The method is simple, and the hydrogel has good biocompatibility and is biodegradable. It has broad application prospects in the field of medical gels.

The above embodiments of the invention are provided for illustrative purposes only. The preferred embodiment does not describe every detail, nor is it limited to the specific implementation method described. According to the content of this manual, it is clear that many modifications and changes can be made. This manual selects and describes these embodiments in detail to better explain the principle and practical application of the invention, enabling technical personnel to understand and use it effectively. The invention is limited only by the claim and its full scope and equivalents.

What is claimed is:

1. A dual antibacterial hemostatic bio-based polyurethane hydrogel containing natural antibacterial components, comprising the following raw materials parts by weight: 40.0-60.0 parts of biodegradable polyester polyol, 40.0-60.0 parts of polyethylene glycol, 24.9 parts of bio-based diisocyanate, 1.3 parts of a hydrophilic alcohol chain extender, 0.1-3.7 parts of curcumin, 2.0-2.5 parts of glycerol, 180.0-200.0 parts of deionized water, and 20.0 parts of a Panax notoginseng extract.

2. The dual antibacterial hemostatic bio-based polyurethane hydrogel containing the natural antibacterial components according to claim 1, wherein the biodegradable polyester polyol is at least one of biodegradable polycarbonate diol, biodegradable polylactic acid diol, and biodegradable polycaprolactone diol; and a number-average molecular weight of the biodegradable polyester polyol is 1000-3000 g/mol.

3. The dual antibacterial hemostatic bio-based polyurethane hydrogel containing the natural antibacterial components according to claim 1, wherein a number-average molecular weight of the polyethylene glycol is 1000-3000 g/mol.

4. The dual antibacterial hemostatic bio-based polyurethane hydrogel containing the natural antibacterial components according to claim 1, wherein the bio-based diisocyanate is at least one of lysine diisocyanate and pentamethylene diisocyanate.

5. The dual antibacterial hemostatic bio-based polyurethane hydrogel containing the natural antibacterial components according to claim 1, wherein the hydrophilic alcohol chain extender is a compound with the following structural formula comprising a hydrophilic group: 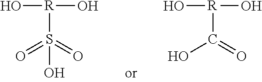
wherein R is a linear or branched alkyl of C3-C4.

6. The dual antibacterial hemostatic bio-based polyurethane hydrogel containing the natural antibacterial components according to claim 1, wherein the curcumin has the following structure:

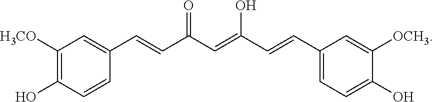 [SMILES: COc1cc(/C=C/C(=O)/C=C(O)/C=C/c2ccc(O)c(OC)c2)ccc1O]

7. The dual antibacterial hemostatic bio-based polyurethane hydrogel containing the natural antibacterial components according to claim 1, wherein the Panax notoginseng extract is composed of 5.0-10.0% Panax notoginseng saponins and 90.0-95.0% deionized water.

8. A preparation method of the dual antibacterial hemostatic bio-based polyurethane hydrogel containing the natural antibacterial components according to claim 1, comprising the following steps:
(1) drying the biodegradable polyester polyol and the polyethylene glycol at 110-130° C. and −0.1 MPa for 1-2 h, and then cooling to room temperature and sealing for storage to obtain a dehydrated biodegradable polyester polyol and a dehydrated polyethylene glycol;
(2) mixing the dehydrated biodegradable polyester polyol and the dehydrated polyethylene glycol obtained in the step (1) with the hydrophilic alcohol chain extender, the curcumin, and the glycerol, and then stirring at 70-90° C. for 1-2 h with a stirring speed of 200-500 rpm to obtain a uniform mixture;
(3) adding the bio-based diisocyanate to the uniform mixture prepared in the step (2), and stirring at 70-90° C. for 2-4 h with a stirring speed of 200-500 rpm to obtain a bio-based polyurethane slurry;
(4) solidifying the bio-based polyurethane slurry prepared in the step (3) in a vacuum oven at 80-100° C., and then immersing in deionized water to obtain a bio-based polyurethane hydrogel;
(5) immersing the bio-based polyurethane hydrogel obtained in the step (4) in liquid nitrogen for 10-15 min, and then vacuum freeze-drying at −20-40° C. for 24-48 h to obtain a porous bio-based polyurethane hydrogel; and
(6) immersing the porous bio-based polyurethane hydrogel obtained in the step (5) in the Panax notoginseng extract for 3 h, so that the Panax notoginseng extract is dispersed in a matrix, and then vacuum freeze-drying at −20-40° C. for 24-48 h to obtain the dual antibacterial hemostatic bio-based polyurethane hydrogel containing the natural antibacterial components.

9. A preparation method of wound dressings, comprising using the dual antibacterial hemostatic bio-based polyurethane hydrogel containing the natural antibacterial components according to claim 1.

10. The preparation method according to claim 8, wherein in the dual antibacterial hemostatic bio-based polyurethane hydrogel containing the natural antibacterial components, the biodegradable polyester polyol is at least one of biodegradable polycarbonate diol, biodegradable polylactic acid diol, and biodegradable polycaprolactone diol; and a number-average molecular weight of the biodegradable polyester polyol is 1000-3000 g/mol.

11. The preparation method according to claim 8, wherein in the dual antibacterial hemostatic bio-based polyurethane hydrogel containing the natural antibacterial components, a number-average molecular weight of the polyethylene glycol is 1000-3000 g/mol.

12. The preparation method according to claim 8, wherein in the dual antibacterial hemostatic bio-based polyurethane hydrogel containing the natural antibacterial components, the bio-based diisocyanate is at least one of lysine diisocyanate and pentamethylene diisocyanate.

13. The preparation method according to claim 8, wherein in the dual antibacterial hemostatic bio-based polyurethane hydrogel containing the natural antibacterial components, the hydrophilic alcohol chain extender is a compound with the following structural formula comprising a hydrophilic group: 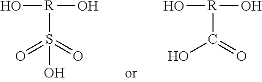
wherein R is a linear or branched alkyl of C3-C4.

14. The preparation method according to claim 8, wherein in the dual antibacterial hemostatic bio-based polyurethane hydrogel containing the natural antibacterial components, the curcumin has the following structure:

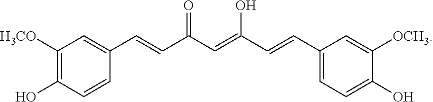 [SMILES: COc1cc(/C=C/C(=O)/C=C(O)/C=C/c2ccc(O)c(OC)c2)ccc1O]

15. The preparation method according to claim 8, wherein in the dual antibacterial hemostatic bio-based polyurethane hydrogel containing the natural antibacterial components, the Panax notoginseng extract is composed of 5.0-10.0% Panax notoginseng saponins and 90.0-95.0% deionized water.

16. The preparation method according to claim 9, wherein in the dual antibacterial hemostatic bio-based polyurethane hydrogel containing the natural antibacterial components, the biodegradable polyester polyol is at least one of biodegradable polycarbonate diol, biodegradable polylactic acid diol, and biodegradable polycaprolactone diol; and a number-average molecular weight of the biodegradable polyester polyol is 1000-3000 g/mol.

17. The preparation method according to claim 9, wherein in the dual antibacterial hemostatic bio-based polyurethane hydrogel containing the natural antibacterial components, a number-average molecular weight of the polyethylene glycol is 1000-3000 g/mol.

18. The preparation method according to claim 9, wherein in the dual antibacterial hemostatic bio-based polyurethane hydrogel containing the natural antibacterial components, the bio-based diisocyanate is at least one of lysine diisocyanate and pentamethylene diisocyanate.

19. The preparation method according to claim 9, wherein in the dual antibacterial hemostatic bio-based polyurethane hydrogel containing the natural antibacterial components, the hydrophilic alcohol chain extender is a compound with the following structural formula comprising a hydrophilic group: 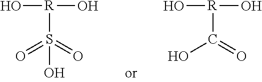
wherein R is a linear or branched alkyl of C3-C4.

20. The preparation method according to claim 9, wherein in the dual antibacterial hemostatic bio-based polyurethane hydrogel containing the natural antibacterial components, the curcumin has the following structure: 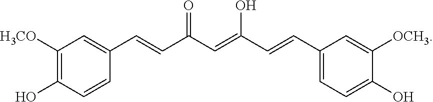 [SMILES: COc1cc(/C=C/C(=O)/C=C(O)/C=C/c2ccc(O)c(OC)c2)ccc1O]
